(19) United States
(12) Patent Application Publication
(10) Pub. No.: US 2005/0220903 A1
(43) Pub. Date: Oct. 6, 2005

(54) Method for processing ginseng by ultra-high pressure

(76) Inventors: Jae Ho Lee, Seoul (KR); Ji Yong Park, Gyeonggi-do (KR); Byong Ki Kim, Gyeonggi-do (KR)

Correspondence Address: JONES DAY, 222 EAST 41ST ST, NEW YORK, NY, 10017, US

(21) Appl. No.: 10/513,977

(22) PCT Filed: May 9, 2003
(86) PCT No.: PCT/KR03/00916

§ 371 Date: Apr. 8, 2005

(51) Int. Cl. A61K 35/78

(52) U.S. Cl. 424/728

(57) ABSTRACT

The present invention relates to a method for increasing active ingredient of Ginseng by Ultra-high pressure. More precisely the invention relates to a method for increasing high active ingredients like ginsenosides comprising treating Ginseng with ultra-high pressure.

### TECHNICAL FIELD

The present invention relates to enhancing active ingredients of Ginseng by using ultra-high pressure technique. In particular, the invention relates to a method for processing fresh Ginseng under high pressure to enhance active ingredients such as ginsenosides.

### BACKGROUND OF ART

Ginseng is a typical nutrition supplement that has been widely used from early ages. In general, Ginseng is classified depending on the manufacturing processes as follows: Fresh Ginseng is obtained from cultivation; White Ginseng is obtained by drying fresh Ginseng in room temperature; and Red Ginseng is obtained by steaming fresh Ginseng at high temperature (98˜100° C.).

Among different Ginsengs mentioned above, the Red Ginseng, which classifies in high-quality product, was produced in purpose of extending storage time in the past. However, recent studies revealed that during the steaming process for producing Red Ginseng, chemical change of the ginsenoside occurs to produce a unique type of ginsenoside; and, further studies are done on the properties of the newly formed compounds. According to the studies and reports published to the present on the properties and efficacy of Red Ginseng, it is good for aging suppression, anti-arteriosclerosis, arteriosclerosis improvement, liver function accentuation, radiation sickness elimination, immune system reinforcement, brain function accentuation, anti-stress, lowering blood sugar, lowering blood pressure, anti-cancer, and many other. However, the active ingredients of Ginseng such as ginsenoside, and unique taste and smell could be lost with the effluence produced during the steaming process.

For the reason, there had been need of a method for producing Ginseng comprising unique ginsenoside of Red Ginseng, thereby enhancing the active ingredients.

### DETAILED DESCRIPTION OF THE INVENTION

The present invention was contrived in need for the solution of the said problems. The objects of the invention are to enhance active ingredient of Ginseng and provide a method for processing Ginseng comprising unique ginsenoside of Red Ginseng.

In order to accomplish the said objects, the invention provides a method for processing Ginseng using ultra-high pressure processing apparatus, wherein the processing is done under the pressure of 200 to 1200 Mpa and at 40 to 90° C. for 5˜60 minutes in time.

It is much desired, according to the method of the present invention, so as to process fresh Ginseng under the pressure of 600˜800 Mpa, at 60˜90° C. for 5˜30 minutes.

The invention method further comprises: pretreatment process of fresh Ginseng using vapor or 60 to 100° C. water for 5˜60 minutes; and pH of the water inside the ultra-high pressure processing apparatus is lowered to the pH 2˜6.

### BRIEF DESCRIPTION OF THE DRAWINGS

FIG. 1 is a graph that shows an analysis on High Performance Liquid Chromatography (hereinafter referred to ‘HPLC’) result of ginsenoside obtained from general Red Ginseng;

FIG. 2 is a graph that shows an analysis on HPLC result of ginsenoside obtained by processing fresh 4-year old Ginseng under ultra-high pressure;

FIG. 3 is a graph that shows an analysis on HPLC result of ginsenoside obtained by fresh 6-year old Ginseng under ultra-high pressure;

FIG. 4a is a graph that shows an analysis on HPLC result of ginsenoside obtained by processing fresh 6-year old Ginseng under ultra-high pressure for 30 minutes; and FIG. 4b is a graph that shows an analysis on HPLC result of ginsenoside obtained by processing fresh 6-year old Ginseng under ultra-high pressure for 60 minutes;

FIG. 5a is a graph that shows an analysis on HPLC result of ginsenoside obtained by processing a 6-year old Ginseng under ultra-high pressure with vacuum packaging; and FIG. 5b is a graph that shows an analysis on HPLC result of ginsenoside obtained by processing a 6-year old Ginseng under ultra-high pressure without packaging;

FIG. 6a is a graph that shows an analysis on HPLC result of ginsenoside obtained by processing fresh 6-year old Ginseng under ultra-high pressure together with steaming; and FIG. 6b is a graph that shows an analysis on HPLC result of ginsenoside obtained by processing fresh 6-year old Ginseng under ultra-high pressure.

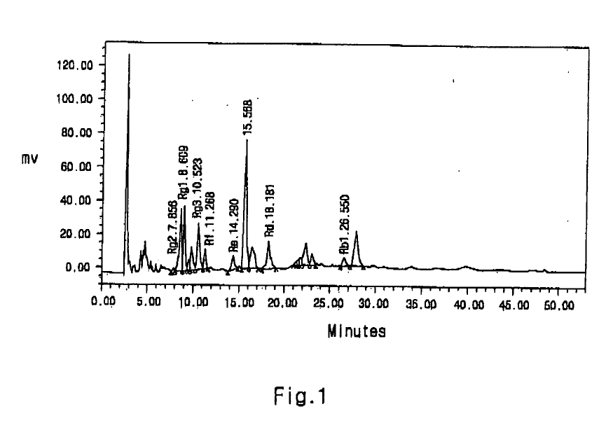

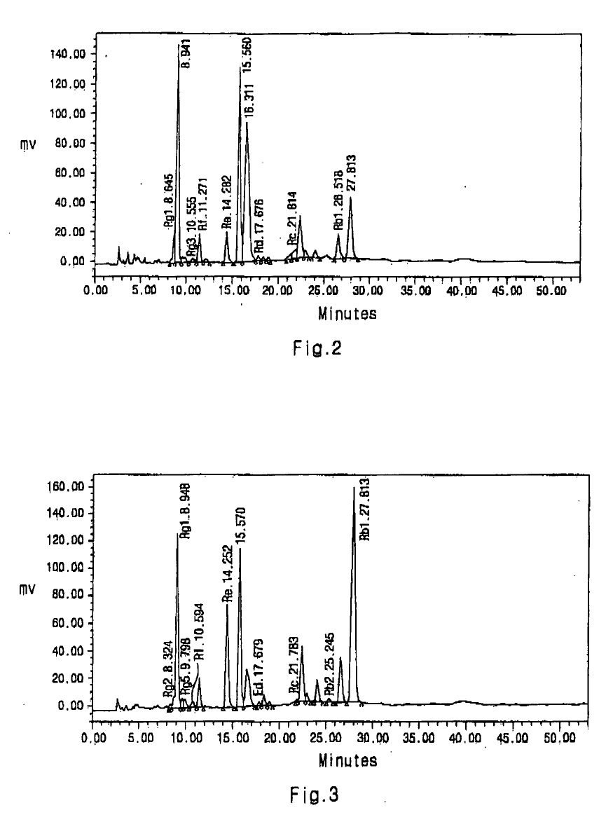

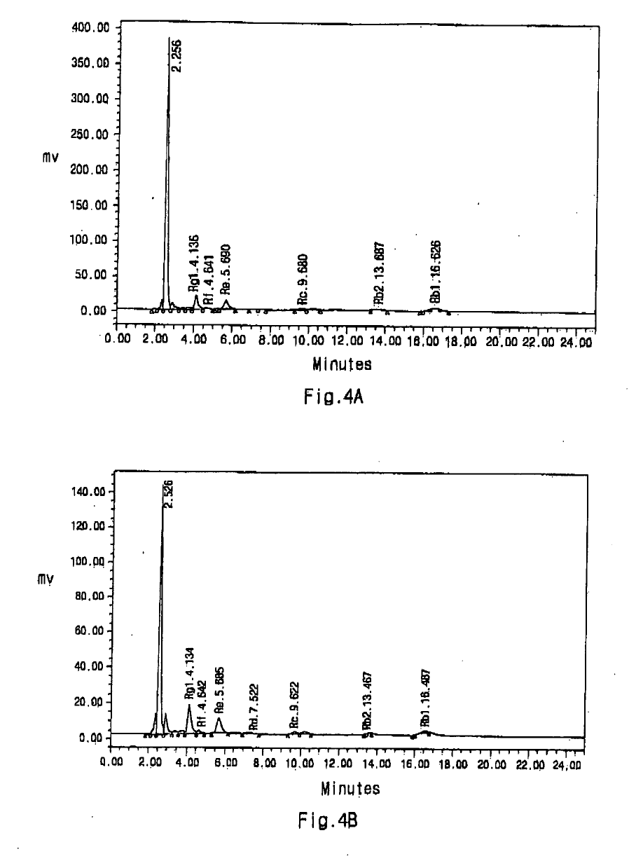

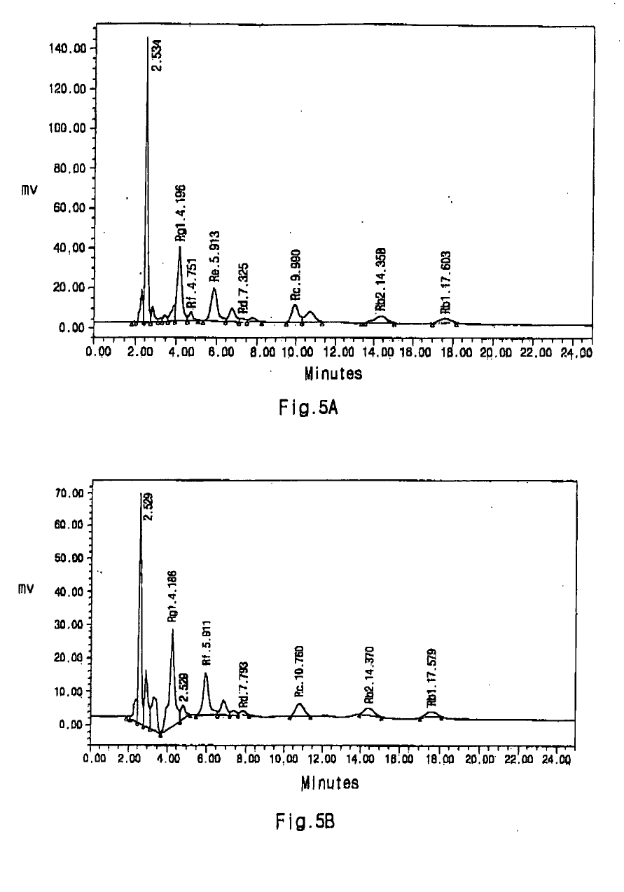

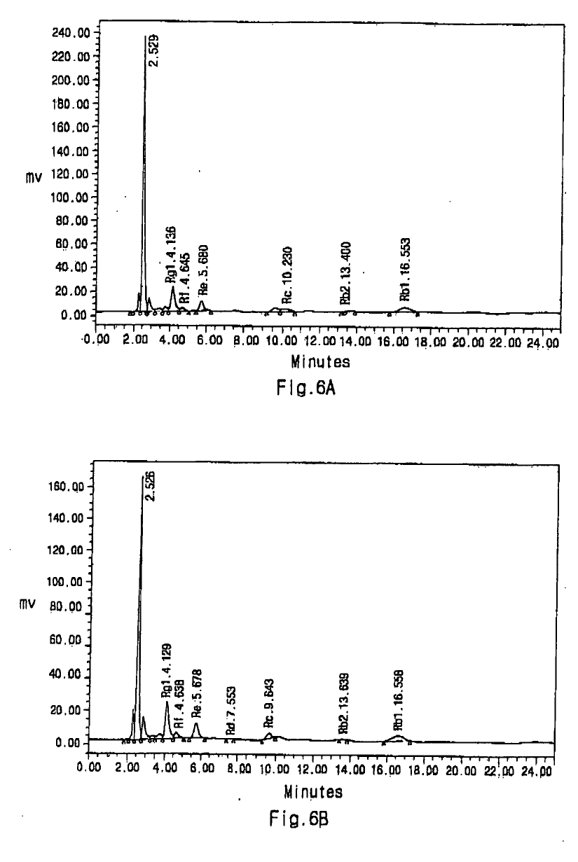

### EXAMPLES

The present invention is described in detail through Examples. Nonetheless, such examples are not restricting the invention.

### Example 1

A 6-year old, by the standard grading of Korea Ginseng Public Enterprise, Ginseng was selected as a sample. The Comparative Example was prepared by removing fine roots of the selected Ginseng, followed by processing under ultra-high pressure (600 to 800 Mpa) at the elevated temperature of 60˜80° C. for 5 to 30 minutes. The processed Ginseng is then cooled to room temperature and is dried by which 12˜13% moisture remains. Meanwhile, the Control Group was prepared by removing fine roots of the selected 6-year old Ginseng, followed by cleansing and steaming at 90˜110° C. temperature for 1 hour. It was then kept at the same temperature for 6 hours, and then steaming process was stopped. The steamed Ginseng was cooled to room temperature, and was dehydrated to produce dehydrated Red Ginseng (12˜13% moisture). Each sample was compared in their function, visible color, and tissue condition by the panel test. The results are summarized in Table 1 below. We find the method for preparing Comparative Example, which was processed by the ultra-high pressure, superior in its quality than the method for preparing steamed Control Group.

TABLE 1
Experiment on quality change depending on each
method of Comparative Example and Control Group

| Name | Comparative Example | Control Group |
|---|---|---|
| Smell | Strong smell of | Weak smell of Ginseng |
| | Ginseng | |
| Taste | Rich and gentle taste | Weak taste of Ginseng |
| | of Ginseng | |
| Visible Color | Transparent, reddish | Dark brown with white |
| | brown with less white | coating |
| | coating | |
| Tissue | Less inner space, | Relatively large inner |
| | fissure occurrence | space and fissure |
| | and more tightly | occurrence |
| | bonded | |

### Example 2

According to Example 1, the Comparative Example was prepared by processing a 4-year old Ginseng under ultra-high pressure, while the Control Group was prepared by steaming a 6-year old Ginseng. Each sample was analyzed in their total crude saponin and various ginsenoside contents. The results are summarized in Table 2 below.

Even though the 4-year old Ginseng was used for the Comparative Example, the total amount of crude saponin was similar to that of the 6-year old Control Group Ginseng. Moreover, the Comparative Example contained unique ginsenoside found in Red Ginseng.

TABLE 2
Experiment on ginsenoside content depending on
each method of Comparative Example and Control Group

(Unit: mg/g)

| | | Comparative Example | Control Group |
|---|---|---|---|
| Crude Saponin | | 21.6 | 22.8 |
| Ginsenoside | -Rg3 | 0.07 | 0.29 |
| | -Rg2 | 0.20 | 0.04 |
| | -Rg1 | 0.22 | 0.35 |
| | -Rf | 0.23 | 0.18 |
| | -Re | 0.22 | 0.14 |
| | -Rd | 0.09 | 0.30 |
| | -Rc | 0.13 | 0.09 |
| | -Rb2 | 0.12 | 0.04 |
| | -Rb1 | 0.36 | 0.20 |
| | -Rh | Confirmed | Confirmed |

### Example 3

According to Example 1, the Comparative Example was prepared by processing a 6-year old Ginseng under ultra-high pressure, meanwhile the Control Group was prepared by steaming a 6-year old Ginseng. Each sample was analyzed in their total crude saponin and various ginsenoside contents. The results are summarized in Table 3 below.

Comparing the total amount of crude saponin, the Comparative Example showed more than twice as much as the Control Group. Furthermore, each amount of ginsenoside contained in the Comparative Example showed more amount in all contents except for −Rg3 and Rd.

TABLE 3
Experiment on ginsenoside content depending on
each method of Comparative Example and Control Group

(Unit: mg/g)

| | | Comparative Example | Control Group |
|---|---|---|---|
| Crude Saponin | | 45.81 | 22.8 |
| Ginsenoside | -Rg3 | 0.21 | 0.29 |
| | -Rg2 | 0.12 | 0.04 |
| | -Rg1 | 1.33 | 0.35 |
| | -Rf | 0.21 | 0.18 |
| | -Re | 1.03 | 0.14 |
| | -Rd | 0.17 | 0.30 |
| | -Rc | 0.12 | 0.09 |
| | -Rb2 | 0.18 | 0.04 |
| | -Rb1 | 2.30 | 0.20 |
| | -Rh | Confirmed | confirmed |

As mentioned in the said Example, the method used in preparing the Comparative Example is superior in its function and saponin content to that of the Control Group.

### Example 4

A 6-year old Ginseng was selected as a sample. The fine roots of the selected Ginseng were removed, followed by processing under ultra-high pressure (600 to 800 Mpa) at the elevated temperature of 60˜80° C. for each 30 and 60 minutes. The separately processed Ginsengs are then cooled to room temperature and dried. Analysis was done on total saponin content and ginsenoside content on each product. The results are summarized in Table 4 below.

TABLE 4
Experiment on ginsenoside content depending on 30
and 60 minutes of processing time

(Unit: mg/g)

| | | Processing Time | Processing Time |
|---|---|---|---|
| | | (30 min.) | (60 min.) |
| Crude Saponin | | 43.50 | 36.10 |
| Ginsenoside | -Rg1 | 2.98 | 1.97 |
| | -Rf | 0.36 | 0.32 |
| | -Re | 4.27 | 2.49 |
| | -Rd | 0.07 | 0.12 |
| | -Rc | 1.23 | 0.39 |
| | -Rb2 | 0.75 | 0.08 |
| | -Rb1 | 4.50 | 0.49 |

As mentioned in the said Example, the 30 minute processing group contained more amount of crude saponin and each ginsenoside content than that of 60 minute processing group.

### Example 5

A 6-year old Ginseng was selected as a sample. The fine roots of the selected Ginseng were removed, followed by processing under ultra-high pressure (600 to 800 Mpa) at the elevated temperature of 60˜80° C. for 30 minutes. At this time, the Comparative Example was prepared under ultra-high pressure without vacuum packaging, while the Control Group was prepared under ultra-high pressure with individual vacuum packaging. When comparison was made on their physical appearance and functions, both total amount of saponin and each ginsenoside contents showed superior results on the vacuum packaged processing. The results are summarized in Table 5 below.

TABLE 5
Experiment on ginsenoside content depending on
each method of Comparative Example and Control Group

(Unit: mg/g)

| | | Comparative Example | Control Group |
|---|---|---|---|
| Crude Saponin | | 22.26 | 37.13 |
| Ginsenoside | -Rg1 | 3.50 | 5.91 |
| | -Rf | 0.75 | 1.07 |
| | -Re | 3.49 | 7.60 |
| | -Rd | 0.17 | 0.33 |
| | -Rc | 1.38 | 3.44 |
| | -Rb2 | 0.97 | 3.06 |
| | -Rb1 | 0.98 | 2.47 |

### Example 6

A 6-year old Ginseng was selected as a sample. The fine roots of the selected Ginseng were removed, followed by processing under ultra-high pressure (600 to 800 Mpa) at the elevated temperature of 60˜80° C. for 5˜60 minutes. At this time, water that fills in the said processing apparatus was treated with citric acid to lower the pH at 6, 4, and 2 before processing Ginseng under ultra-high pressure. Comparing the unique ginsenoside Rg content, more amount results with lower pH accordingly.

### Example 7

6-year old Ginseng was selected as a sample. The fine roots of the selected Ginseng were removed, followed by processing under ultra-high pressure (600 to 800 Mpa) at the elevated temperature of 60˜80° C. for 30 minutes. At this time, the Comparative Example was prepared under ultra-high pressure after vacuum packaging, while the Control Group was prepared further comprising the step of steaming fresh Ginseng before vacuum packaging. When comparison was made on their physical appearance and functions, the Control Group showed more amount in total saponin content, and each amount of ginsenoside contained in the Control Group showed more amount in contents except for Re, Rf, Rg1. The results are summarized in Table 7 below.

TABLE 7
Experiment on ginsenoside content depending on
each method of Comparative Example and Control Group

(Unit: mg/g)

| | | Comparative Example | Control Group |
|---|---|---|---|
| Crude Saponin | | 28.00 | 38.20 |
| Ginsenoside | -Rg1 | 3.20 | 3.00 |
| | -Rf | 0.91 | 0.74 |
| | -Re | 2.88 | 2.50 |
| | -Rd | 0.06 | Confirmed |
| | -Rc | 1.26 | 1.27 |
| | -Rb2 | 0.10 | 0.41 |
| | -Rb1 | 3.34 | 4.33 |

### INDUSTRIAL AVAILABILITY OF THE INVENTION

The present invention uses ultra-high pressure comprising the said components to enhance active ingredient about twice that of prior method for processing Ginseng.

1. A method for processing Ginseng using ultra-high pressure processing apparatus, wherein the processing is done under the pressure of 200 to 1200 Mpa and the temperature of 40 to 90° C. for 5˜60 minutes.

2. The method according to claim 1, further comprises pretreatment process on a fresh Ginseng with steam or 60˜100° C. water.

3. The method according to claim 1, wherein a fresh Ginseng is Vacuum packaged before processing Ginseng under ultra-high pressure.

4. The method according to claim 1, wherein pH of water inside the said processing apparatus is lowered to pH2˜6 before processing Ginseng under ultra-high pressure.
